(19) United States
(12) Patent Application Publication
(10) Pub. No.: US 2024/0016994 A1
(43) Pub. Date: Jan. 18, 2024

(54) SURGICAL DRAINAGE DEVICE

(71) Applicants: UNIVERSITA' DEGLI STUDI DI UDINE, Udine (IT); AZIENDA SANITARIA UNIVERSITARIA FRIULI CENTRALE, Udine (IT)

(72) Inventors: Vittorio Alessandro CHERCHI, Udine (IT); Umberto BACCARANI, Udine (IT); Andrea RISALITI, Udine (IT)

(21) Appl. No.: 18/256,762

(22) PCT Filed: Dec. 7, 2021
(86) PCT No.: PCT/IT2021/050396

§ 371 Date: Jun. 9, 2023

(30) Foreign Application Priority Data
Dec. 10, 2020 (IT) 102020000030308

(51) Int. Cl. A61M 1/00

(52) U.S. Cl. CPC A61M 1/84; A61M 2202/0403

(57) ABSTRACT

Surgical drainage device (10) comprising a first tubular body (11) inside which a second tubular body (12) is partly inserted in a telescopic and sliding manner.

### FIELD OF APPLICATION

The present invention relates to a surgical drainage device of an organic fluid from a drainage zone to the outside of a patient's body.

In particular, the surgical drainage device of the present invention is used in bile duct, resective and liver transplantation surgery. In this case, its function is to drain the bile produced by the liver out of the body.

The present surgical device may also be used in other surgeries, for example in neurosurgery, in performing craniotomies, or in esophageal surgery or other surgery.

### BACKGROUND ART

Surgical drainage devices used, for example but not limited to, in bile duct, resective and liver transplantation surgery are known.

There are conditions, including injury or the presence of a suture or anastomosis on the bile duct, wherein the bile must be derived from the bile duct without passing through it as it could leak out and spread to the abdomen causing severe complications.

The so-called Kehr tube, a surgical instrument in the shape of a “T” invented by Hans Kehr, which is used to drain the bile produced by the liver to the outside of the body and which at the same time allows doctors to monitor its quantity and quality, is known. The “T” shape of the Kehr tube is necessary for the area where it must be placed: the choledochus. In fact, the bile duct is a generic term for the duct that, from inside the liver, leads the bile to the duodenum. Along this path, the bile duct takes different names depending on the anatomical region it crosses; intrahepatic ducts, extrahepatic bile ducts, common hepatic duct and choledochus duct below the cystic duct outlet. The Kehr tube is, in particular, one of the most used traditional drainage devices in this type of surgery and includes an oblong central tubular body and a shorter peripheral tubular branch, fluidly connected to one end of the central tubular body so as to form a “T”-shaped tube overall.

The peripheral tubular branch is connected, with respect to its centreline, to the central tubular body so that two arms are defined.

This “T”-type drainage device is made of flexible material and in a single body or in any case in such a way that the central tubular body and the peripheral tubular branch are mutually arranged in a fixed and immovable position, despite the flexibility guaranteed by the material.

The peripheral tubular branch, which forms the “T” arm, is positioned within the bile duct while the central tubular body is able to escape, through an incision, from the patient's body to allow the drainage of the bile, for example inside a special collection bag.

In the case of liver transplant surgery, the “T”-shaped drainage device is inserted shortly before the bile duct of the donor and the recipient join. Anastomosis is performed by suturing the entire circumference of the bile ducts of the donor and recipient.

This suture line represents an area of lower resistance often subject to complications such as partial or total rupture of the anastomosis with leakage of the bile into the abdomen.

If there are concerns regarding the tightness of the surgical sutures, the “T”-shaped drainage device allows to wait for the time needed for the continuity of the biliary tract to be restored, preventing the aforementioned complications.

After a certain variable time from its positioning—usually within a few months—the device has to be removed.

A disadvantage of the traditional “T”-shaped drainage device is that the “T” shape is an obstacle to its extraction from the bile duct that occurs by manual traction and requires a certain force from the surgeon.

In fact, the peripheral tubular branch, arranged parallel to the development of the bile duct, must pass through the small aperture on the wall of the bile duct through which the central tubular body exits and which has a passage gap equal to the external diameter of the latter.

During the removal procedure, the aforementioned device, which is flexible and elastic, stretches and tightens. The tensile forces force the arms of the peripheral tubular branch to fold to engage the aforementioned aperture. Where only the central tubular body used to pass, the two folded arms of the peripheral tubular branch must now pass.

The energetic tractions required to extract such a device can cause injury from rupture/splitting of involved structures and numerous complications including biliary leaks, biliary peritonitis, biliomas, sepsis, and postoperative biliary stenosis.

Document US-A-2020/016382 and document US-A-2003/149395 both describe Y-shaped drainage devices specifically made for intrahepatic bile drainage. They provide two tubes, sliding one inside the other, for the purpose of draining the bile out of the body. Therefore, such known devices, by virtue of their “Y” shape, cannot be introduced into the common extrahepatic duct and choledochus, which differ from the intrahepatic ones in that they are unique and of greater size. The “Y” shape of these known devices is designed to be located within the branches of the intrahepatic bile duct and at the first-order extrahepatic limit (right-left). They could not be used inside the choledochus as the “T”-shaped Kehr tube. By placing, within the choledochus, a known Y-shaped device, the arms of the device would be forced into a position transverse to the axis of the duct. Forces of this type would create injuries/pressure perforations of the arms on the walls of the duct.

Such known devices are designed for the drainage of bile within the liver or in its first extrahepatic bifurcation (right-left bifurcation). In fact, the bile duct is similar in structure to the branching of a tree. The biliary structure of the liver is also called the “biliary tree”. As for a tree, the ramifications are of various orders; from the largest to the smallest. As with trees, the ramifications originate from each other at an angle that is generally open but tends to be acute. Such known devices, in fact, consist of two components that can be angled together elastically to adapt to this type of anatomy. However, such known devices are not suitable to be placed inside a straight and regular duct such as the choledochus where the angle between the components of the “Y” shape could even be harmful and predispose to complications such as bile duct perforation and peritonitis.

The choledochus duct, for which the Kehr tube is used, has a variable length, of about 6-7 cm. At the end of the duct there is a muscle valve that regulates and controls the passage of the bile in the duodenum. In case of blockage of this valve, the bile undergoes a slowdown in the outflow with consequent accumulation within the duct. As for liquids that encounter an obstacle to outflow, they put pressure on the duct inside which they are located or, if necessary, they accumulate increasing in level until they rise up along the duct itself. In the case of liver transplants or bile duct lesions, this valve undergoes a shock that paralyses it (papillary spasm), and that hinders the outflow of the bile as mentioned. This puts pressure on the choledochus duct, shortly after performance of the surgical suture that was performed on the transplant (or on the surgically repaired lesion of the duct). The liver transplant in fact implies, for its success, that the biliary tract is also “reattached” at the level of the choledochus duct. The donor choledochus and recipient choledochus are joined together by a hand-suture with surgical suture thread. In the first days and weeks after transplantation, this represents the true Achilles' heel of the transplant itself, as recognized by the world's scientific literature. Bile leaks through the suture can cause biliary peritonitis and death of the patient. The choledochus at the point where it is sutured is extremely delicate and sensitive to any insult. To be able to repair safely, it must remain as dry as possible and not subject to tension. In practice, the level of bile should not rise and increase the pressure within the duct as in the example of water described.

In this respect, the known “Y”-shaped devices are not designed to perform this function. They are in fact located very far from the point at risk on the choledochus. Due to their conformation and location, they cannot drain the outflow of bile further down, in the last portion of the choledochus duct, towards the valve.

Instead, the T-shaped Kehr tube is inserted into the choledochal reconstruction before the suture with the thread is completed. With the “proximal or upper” arm (the one that goes to the liver), the T-shaped Kehr tube drains the bile produced by the liver that is flowing along the way. With the “distal or lower” arm (the one towards the end of the duct), it drains the bile that has drained and is accumulating at the bottom.

This allows the suture to be kept as dry as possible as it is for external wounds that do not heal when moist or wet.

In addition, the aforementioned known “Y”-shaped devices are provided for biliary insertion and sliding is provided mainly to provide advantages during positioning in place and not for removal at the end of their use.

Moreover, the treatment of the stenosis (narrowing of the biliary tract) for which the aforementioned known devices have been designed, hardly finds any place in modern medical practice. Thanks to the new non-invasive methods, in fact, the treatment of biliary stenosis takes place percutaneously (through the patient's abdominal wall) or endoscopically (ERCP: retrograde endoscopy).

Therefore, there is the need to perfect a surgical drainage device which can overcome at least one of the drawbacks of the known art.

In particular, an object of the present invention is to make such a surgical drainage device which allows to eliminate, or at least reduce, the problems related to its extraction from the patient's body.

A further object of the present invention is to make such a surgical drainage device which is very easy to remove and which requires much lower tensile stresses than those required for the extraction of a traditional drainage device.

The Applicant has studied, tested and realized the present invention to overcome the drawbacks of the prior art, and to obtain these and further objects and advantages.

### DISCLOSURE OF THE INVENTION

The present invention is expressed and characterized in the independent claim. The dependent claims show other features of the present invention or variants of the main solution idea.

In accordance with the aforementioned purposes, a surgical drainage device that overcomes the limits of the known art and eliminates the defects present therein comprises a first tubular body having a first lateral surface having a first passage aperture. A second tubular body is at least partly inserted in a telescopic and sliding manner into said first tubular body through the first passage aperture. The second tubular body has a second lateral surface having a second passage aperture configured to allow fluidic communication between the interior of the first tubular body and the interior of the second tubular body.

According to an aspect, the second tubular body is able to slide within the first tubular body so that a respective second terminal portion of the second tubular body is able to protrude beyond the first passage aperture, selectively branching off with respect to a corresponding first terminal portion of the first tubular body.

According to one aspect, the first tubular body is a distinct and separate component with respect to the second tubular body.

According to another aspect, the first passage aperture and the second passage aperture respectively divide the first tubular body and the second tubular body into two respective oblong portions and into the aforementioned first and second terminal portions.

According to another aspect, in a first operating condition, the aforementioned second terminal portion is arranged outside the first tubular body through the first passage aperture defining together with the first terminal portion an end bifurcation that gives the device a “T” conformation. In a second operating condition, the first passage aperture is free, that is, it is not engaged by the second tubular body.

In particular, according to an aspect of the invention, the aforementioned device is configured to be introduced into the common extrahepatic duct and choledochus for bile drainage.

Unlike the above-mentioned documents, which provide for “Y”-shaped devices, therefore unsuitable for carrying out the functions of a traditional Kehr tube, this invention provides for a “T” shape such as the Kehr tube itself, to fulfil the specific functions and applications, however overcoming the disadvantages related to the removal from the patient's body, by virtue of the sliding and telescopic structure described above. In particular, the device described here is “T”-shaped but provides, unlike the traditional Kehr tube, that the aforementioned tubular bodies are sliding and therefore decomposable, allowing to avoid dangers for the patient deriving from the removal, allowing an easy removal of the instrument from the biliary tract reducing the risk of a biliary fistula and peritonitis in the patient.

Accordingly, the present invention retains the specific functions of the so-called Kehr tube, typically used for the drainage of bile in liver, bile duct and liver transplant surgery, additionally overcoming the problems and criticalities of the traditional Kehr tube, generally related to the fact that its use can lead to severe complications especially during its removal. The present invention, therefore, makes it possible to avoid these complications with considerable advantages for patient safety.

The present invention in the form of a “T”, therefore, is particularly suitable for positioning in the extrahepatic and choledochus duct for the drainage of the bile, which duct has a variable length, of about 6-7 cm. The device described here, due to its anatomical shape and size (up to about 3 mm), is safer and can only be used in the extrahepatic and choledochal duct, having a regular and straight preformed “T” shape.

The device described herein, thanks to its “T” shape, is suitable to be inserted inside the reconstruction of the choledochus, before the suture with the thread is completed. With the “proximal or upper” arm, which goes to the liver, the device drains the bile produced by the liver that is flowing along the way, while with the “distal or lower” arm, towards the end of the duct, it drains the bile that has drained and is accumulating at the bottom.

This allows the suture to be kept as dry as possible as it is for external wounds that do not heal when moist or wet.

Moreover, the device described here, by virtue of the sliding between the tubular bodies, has undoubted advantages in the removal at the end of its use. For this reason, the device described here has a preformed “T” shape, that is, it consists of two angled components that allow the device to remain “fixed” in place and to proceed to the extraction of the tubular components one at a time without traction and/or trauma.

The present invention differs substantially from the known “Y”-shaped devices in that it has a T shape, which can be decomposed thanks to the sliding of the tubular components, which T shape keeps it in place while it stays in the choledochus. The device described herein allows complete drainage of bile from within the choledochus after surgical repair of lesions and after liver transplantation. Its sliding mechanism allows easy and safe removal from the choledochus. On the contrary, the known “Y”-shaped devices cannot be placed inside the choledochus, under penalty of serious pressure injuries and insufficient drainage of the bile. Even the removal of the known “Y” shaped devices, if placed in the choledocus, would cause tear injuries due to the rectilinearization that they would undergo at the time of their removal even if their parts were extracted individually.

On the contrary, the device described herein with a “T” shape is particularly suitable for the treatment of lesions of the choledochus or after its reconstruction during a liver transplant and it has been found that, for this task, its operating mechanism proves to be irreplaceable.

According to another aspect, in the first operating condition, the first terminal portion and the second terminal portion diverge from the axis of development of the respective tubular bodies, determining a transverse bulk that is greater than the transverse bulk that the device has in the second operating condition in which the second terminal portion has a configuration aligned both with the respective second oblong portion and with the first oblong portion.

The particular detachable component configuration allows the surgical drainage device to be easily removed from a patient's body once its function has been fulfilled. In fact, by sliding the second tubular body, in particular the second terminal portion, inside the first, it is avoided that the two terminal portions must be forced to pass simultaneously through an access aperture that has a limited diameter. This avoids exerting excessive, and sometimes destructive, stress on the structures of the patient's body, thus preserving their integrity.

Some advantages of the present invention can therefore be:
easy and safe extraction;
simple and functional structure;
reduction of complications related to its removal from the bile duct;
reduction of the need for ERCP (endoscopic retrograde cholangiopancreatography) to follow extraction manoeuvres especially after liver transplant surgery.

### ILLUSTRATION OF THE DRAWINGS

These and other aspects, features and advantages of the present invention will become clear from the following embodiment disclosure, given as a non-limiting example, with reference to the attached drawings in which:

FIG. 1 is a sectional view of a surgical drainage device in accordance with embodiments described herein and in a first operating condition;

FIG. 2 is a sectional view of the device of FIG. 1 in a second operating condition;

FIGS. 3-5 illustrate, for educational purposes, the operational phases steps for removing the device of FIG. 1 from a corpse or the body of a dummy;

FIGS. 6-8 schematically illustrate some possible applications of the device of FIGS. 1-2.

To facilitate understanding, identical reference numbers have been used, where possible, to identify identical common elements in the figures. It is to be understood that elements and features of an embodiment can be conveniently combined or incorporated into other embodiments without further clarification.

### DESCRIPTION OF EMBODIMENTS

Reference will now be made in detail to the possible embodiments of the invention, one or more examples of which are shown in the attached figures by way of non-limiting example. The phraseology and terminology used herein is also for non-limiting exemplary purposes.

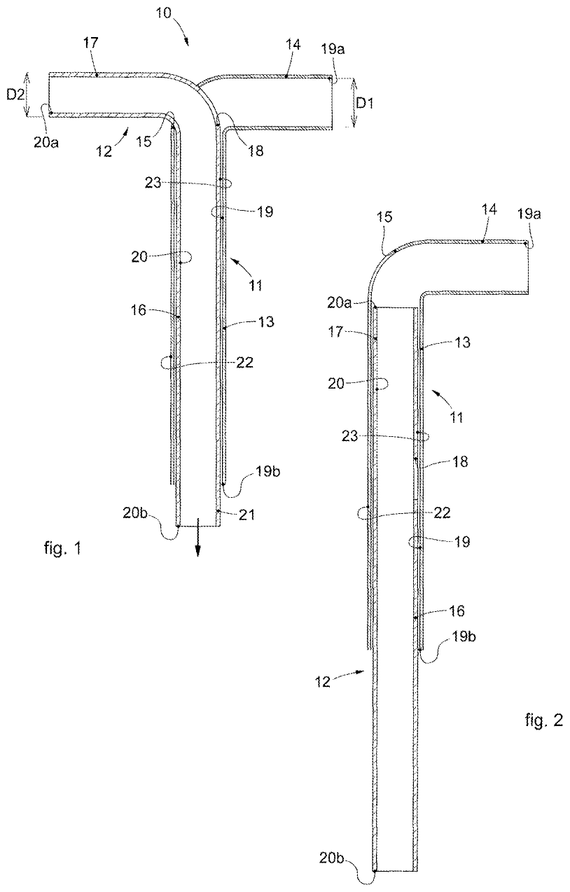

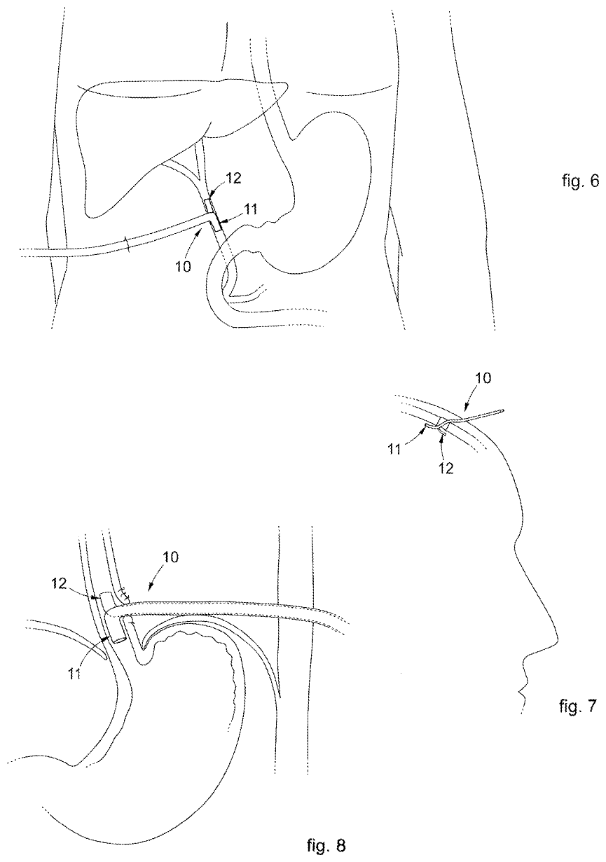

With reference to FIGS. 1-2, a surgical drainage device 10 is described that can be used, for example, in bile duct, resective and liver transplantation surgery for the drainage of an organic fluid, in this case of biliary fluid (FIG. 6) or abdominal fluid, or in neurosurgery to reduce intracranial pressure in case of haemorrhage (FIG. 7), or in esophageal surgery (FIG. 8) or other types of surgery.

The device 10 is configured to be introduced into the common extrahepatic duct and choledochus for bile drainage.

The device 10 comprises a first tubular body 11 having a first lateral surface 22 which has a first passage aperture 15.

Inside the first tubular body 11, a second tubular body 12 is at least partly inserted in a telescopic and sliding manner through the aforementioned first passage aperture 15.

The second tubular body 12 has a second lateral surface 23 having a second passage aperture 18 configured to allow fluidic communication between the interior of the first tubular body 11 and the interior of the second tubular body 12.

The second tubular body 12 is able to slide within the first tubular body 11 so that a respective second terminal portion 17 of the second tubular body 12 is able to protrude beyond the first passage aperture 15, selectively branching off with respect to a corresponding first terminal portion 14 of the first tubular body 11.

The first tubular body 11 is a distinct and separate component with respect to the second tubular body 12.

The device 10 is completely dismountable, that is, the second tubular body 12 can slide inside the first tubular body 11, through the first passage aperture 15, and be possibly extracted from the latter through an outlet aperture 19b of the first tubular body 11.

The first tubular body 11 and the second tubular body 12 are made of flexible and biocompatible material. The flexibility of the material is necessary both for the particular application, the surgical one, and to allow the sliding and the adaptation of the second tubular body 12 inside the first tubular body 11, as will be described in more detail below.

The telescopic and sliding insertion of the second tubular body 12 inside the first tubular body 11 allows, during the removal of the device—after its purpose is fulfilled—to reduce or completely eliminate all the complications encountered during the removal of the traditional drainage devices. In fact, the bulk of the device 10 during removal can be reduced to the bulk of the first tubular body 11 only. This avoids causing excessive stress or traction on the structures concerned, allowing their integrity to be preserved.

The first passage aperture 15 and the second passage aperture 18 respectively divide the first tubular body 11 and the second tubular body 12 into two respective oblong portions 13, 16 and into the aforementioned first and second terminal portions 14, 17.

The first passage aperture 15 has a passage gap substantially equal to the external diameter D2 of the second tubular body 12 so as to allow its passage and sliding, but with limited clearance.

Sealing means configured to prevent organic fluid from flowing through any possible interspace between the first passage aperture 15 and the second tubular body 12 may possibly be associated with the first passage aperture 15.

The device 10 has a collection end, configured to be connected to a bag, a container or other collection device which is, in use, outside the patient's body, and an opposite reception end constituted by the aforementioned terminal portions 14, 17 which are, in use, inside the patient's body, in particular inside the space to be drained, as will be described in more detail below.

The first terminal portion 14 is made in a single body and in continuity with the first oblong portion 13. Similarly, the second terminal portion 17 is also made in a single body and in continuity with the second oblong portion 16. However, it is not excluded that the first tubular body 11 and the second tubular body 12 may consist of several components associated with each other.

The first terminal portion 14 and the second terminal portion 17 have a shorter length than the length of the respective oblong portions 13, 16. For example, the axial length of the first terminal portion 14 and the second terminal portion 17 may be between about 0.1 and about 0.15 times the linear length of the first tubular body 11 and the second tubular body 12, respectively.

The first tubular body 11 and the second tubular body 12 have a first axial channel 19 through and a second axial channel 20 through delimited by respective inlet apertures 19a, 20a and outlet apertures 19b, 20b.

The inlet apertures 19a, 20a delimit on one side the respective terminal portions 14, 17 and through them, in use, the organic fluid that must be drained enters, while the outlet apertures 19b, 20b delimit, on the opposite side, the respective oblong portions 13, 16.

The first axial channel 19 and the second axial channel 20 may have constant section or may have narrowing or widening portions along their development.

With particular reference to FIG. 1, in a first operating condition of the device 10, the second terminal portion 17 is arranged outside the first tubular body 11 defining together with the first terminal portion 14 an end bifurcation that gives the device 10 a “T” shape. In the present case, the first terminal portion 14 and the second terminal portion 17 are folded with respect to the respective oblong portions 13, 16 so that the first tubular body 11 and the second tubular body 12 have an “L” shape.

The first terminal portion 14 and the second terminal portion 17 diverge, by way of example but not necessarily in a specular manner, from the axis of development of the respective tubular bodies 11, 12, determining a transverse bulk that is functional to the uptake necessary for the drainage of the specific area of the body.

The first terminal portion 14 and the second terminal portion 17 may be more or less tilted relative to the central body defined by the first tubular body 11 and the portion of the second tubular body 12 disposed therein. The mutual tilting of the aforementioned terminal portions 14, 17 is defined by the physical limits—walls or other structures—that define the space that must be drained and by the flexibility of the material that constitutes them.

In a possible configuration that the device 10 can have in the first operating condition, the first terminal portion 14 and the second terminal portion 17 are arranged substantially aligned and specular so as to define the aforementioned bifurcation that takes on a real “T” conformation, and the second oblong portion 16 is aligned and inserted coaxially within the first oblong portion 13.

In the first operating condition, the first passage aperture 15 is engaged by a segment of the second tubular body 12, in particular by the second terminal portion 17, and the second passage aperture 18 is inside the first tubular body 11.

In the first operating condition, the second passage aperture 18 is substantially aligned and opposed to the first passage aperture 15. In this way, the first axial channel 19 is fluidly communicating with the second axial channel 20 through the second passage aperture 18. In this way, the organic fluid collected from the first end segment 14 can also flow into the second passage channel 20 to be drained outward.

In the first operating condition, the first passage aperture 15 is made substantially at the outer elbow that the first terminal portion 14 defines with the first oblong portion 13, and, similarly, the second passage aperture 18 is made substantially at the outer elbow that the second terminal portion 17 defines with the second oblong portion 16, FIG. 1.

With reference to FIG. 1, the second oblong segment 16 has a greater length than the first oblong segment 13 defining, in the first operating condition, a gripping portion 21 protruding from the first oblong segment 13 and having a length comprised between about 0.5 cm and about 4 cm, preferably around 1 cm. In particular, the gripping portion 21 is protruding from the outlet aperture 19b delimiting the first oblong portion 13. This gripping portion 21, opposed to the second terminal portion 17, can act as a gripping zone for the traction of the second tubular body 12 allowing the sliding of the second terminal portion 17 inside the first tubular body 11.

With particular reference to FIG. 2, in a second operating condition of the device 10 the first passage aperture 15 is free or is not engaged by the second tubular body 12 and the second terminal portion 17 is arranged inside the first tubular body 11.

In the second operating condition, the transverse bulk of the device 10 is reduced. In fact, the second terminal portion 17 which previously had a transverse direction, now has a configuration aligned both with the respective second oblong portion 16 and with the first oblong portion 13 of the first tubular body 11. This minimal bulk condition allows to greatly reduce the stresses that are caused during the removal of the device 10 from the patient's body.

In the transition from the first operating condition to the second operating condition the second tubular body 12 is forced to slide inside the first tubular body 11 so that the second terminal portion 17 is disposed therein and a part of the second oblong portion 16 exits the outlet aperture 19b delimiting the first oblong portion 13.

In the second operating condition, the second passage aperture 18 is misaligned with respect to the first passage aperture 15 and, in the configuration shown in FIG. 2, is directly facing the wall of the first axial channel 19.

The second operating condition may also comprise the configuration in which the second tubular body 12 is completely extracted from the first tubular body 11 through the aforementioned outlet aperture 19b.

According to embodiments, the external diameter D2 of the second tubular body 12 is slightly smaller than the internal diameter D1 of the first tubular body 11 which defines, moreover, the size of the first axial channel 19. It is advantageous, in fact, that the second tubular body 12 is substantially adherent to the first tubular body 11, but that mutual sliding is guaranteed. In this way, the organic fluid collected from the two terminal portions 14, 17 is conveyed all within the second axial channel 20.

With particular reference to FIGS. 6-8, when the device 10 is in the first condition of use and within the patient's body, the two terminal portions 14, 17 are arranged within the space to be drained, or even drainage space.

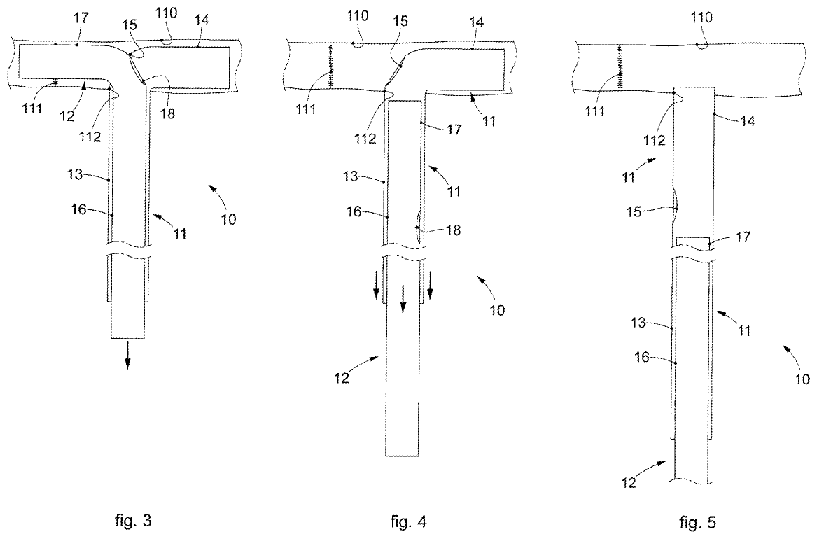

In the case illustrated in FIGS. 3-6, relating to liver transplant surgery, this drainage space is the bile duct choledochus 110 and the device 10 is able to drain the bile fluid out of the body to limit as much as possible contact with the surgical suture 111 that connects the bile duct of the recipient with that of the donor. In FIG. 3, the device 10 is in the first operating condition and the two terminal portions 14, 17 are inserted into the choledochus 110 while the remaining portion of the device 10 exits the choledochus 110 through an access aperture 112 that has the size of the external diameter of the first tubular body 11.

The embodiments illustrated in FIGS. 3-6 show the phases of an educational method for removing the device 10 from a corpse or from the body of a dummy, wherein the device 10, which is in the first operating condition, has the first and second terminal portions 14, 17 within the drainage space. The educational method for removing the device 10 provides at least:

a first extraction phase, FIG. 3-4, in which the second tubular body 12 is pulled from the gripping portion 21 until the second terminal portion 17 disengages the first passage aperture 15 and, for example, is positioned inside the first tubular body 11, and

a second extraction phase, FIG. 5, in which the device 10 is pulled in such a way as to allow the first terminal portion 14 to escape from the drainage space, in this case through the access aperture 112.

It is possible to provide that, in the first extraction phase, the second tubular body 12 is completely extracted from the first tubular body 11 following an extraction direction that initially disengages the first passage aperture 15 and subsequently the outlet aperture 19b of the first axial channel 19.

The described educational method for removal allows to reduce the efforts necessary to extract the device 10 from the specific drainage space. In fact, since the extraction of the second arm 17 takes place by sliding the second tubular body 12 inside the first tubular body 11, an excessive, and sometimes destructive, stress on the structures of the patient's body is avoided.

It is clear that modifications and/or additions of parts or phases may be made to the surgical drainage device described so far, without departing from the scope of the present invention as defined by the claims.

In the following claims, the references in parentheses have the sole purpose of facilitating reading and must not be considered as limiting factors as regards the scope of protection underlying the specific claims.

1. Surgical drainage device comprising a first tubular body having a first lateral surface which has a first passage aperture, inside said first tubular body there being at least partly inserted, in a telescopic and sliding manner, a second tubular body through said first passage aperture, said second tubular body having a second lateral surface which has a second passage aperture configured to allow the fluidic communication between the inside of said first tubular body and the inside of said second tubular body,
wherein said first passage aperture and said second passage aperture respectively divide said first tubular body and said second tubular body into two respective oblong portions and into said first and second terminal portions,
wherein, in a first operating condition, said second terminal portion is arranged outside said first tubular body through said first passage aperture defining together with said first terminal portion an end bifurcation which confers a “T”-shaped conformation upon said device, and
wherein, in a second operating condition, said first passage aperture is free and not engaged by said second tubular body.

2. Device as in claim 1, wherein said second tubular body is able to slide inside said first tubular body so that a respective second terminal portion of said second tubular body is able to protrude beyond said first passage aperture, selectively branching out with respect to a corresponding first terminal portion of said first tubular body.

3. Device as in claim 1, wherein said first tubular body is a distinct and separate component with respect to said second tubular body.

4. Device as in claim 1, wherein in said first operating condition said first terminal portion and said second terminal portion diverge from the axis of development of the respective tubular bodies determining a transverse bulk which is greater than the transverse bulk that said device has in said second operating condition in which said second terminal portion has a configuration aligned both with the respective second oblong portion and also with said first oblong portion.

5. Device as in claim 1, wherein in said first operating condition said first terminal portion and said second terminal portion are disposed substantially aligned and specular, and in that said second oblong portion is inserted coaxially inside said first oblong portion.

6. Device as in claim 1, wherein in said first operating condition, said second passage aperture is aligned with and opposite said first passage aperture.

7. Device as in claim 1, wherein said first passage aperture has a passage gap substantially equal to an external diameter of said second tubular body.

8. Device as in claim 1, wherein said first terminal portion and said second terminal portion have the same length, while said second oblong segment has a greater length than said first oblong segment defining, in said first condition, a gripping portion protruding with respect to said first oblong segment.

9. Device as in claim 1, wherein the external diameter of said second tubular body is slightly smaller than the internal diameter of said first tubular body, so that said second tubular body adheres to said first tubular body, yet their reciprocal sliding is guaranteed.

10. Device as in claim 1, wherein it is configured to be introduced into the common extrahepatic duct and choledochus for bile drainage.

11. Device as in claim 2, wherein said first tubular body is a distinct and separate component with respect to said second tubular body.

12. Device as in claim 11, wherein in said first operating condition said first terminal portion and said second terminal portion diverge from the axis of development of the respective tubular bodies determining a transverse bulk which is greater than the transverse bulk that said device has in said second operating condition in which said second terminal portion has a configuration aligned both with the respective second oblong portion and also with said first oblong portion.

13. Device as in claim 2, wherein in said first operating condition said first terminal portion and said second terminal portion diverge from the axis of development of the respective tubular bodies determining a transverse bulk which is greater than the transverse bulk that said device has in said second operating condition in which said second terminal portion has a configuration aligned both with the respective second oblong portion and also with said first oblong portion.
